(19) United States
(12) Patent Application Publication
(10) Pub. No.: US 2014/0005670 A1
(43) Pub. Date: Jan. 2, 2014

(54) DRILL SYSTEM FOR ACETABULAR CUP IMPLANTS

(71) Applicant: Henry H. Fletcher, Cameron Park, CA (US)

(72) Inventor: Henry H. Fletcher, Cameron Park, CA (US)

(21) Appl. No.: 13/741,156

(22) Filed: Jan. 14, 2013

Related U.S. Application Data
(63) Continuation of application No. 12/069,747, filed on Feb. 13, 2008, now abandoned.
(60) Provisional application No. 60/901,218, filed on Feb. 13, 2007.

(51) Int. Cl. A61B 17/17; A61B 17/16

(52) U.S. Cl. CPC A61B 17/1746; A61B 17/1666
USPC 606/81; 606/96

(57) ABSTRACT

A drill guide system comprising:
a hemispherical cup having an opening therein;
a body having a distal end, a proximal end and a lumen extending between the distal end and the proximal end; and
a connector for connecting the body to the hemispherical cup so that the lumen at the distal end of the body is aligned with the opening in the hemispherical cup.

### REFERENCE TO PENDING PRIOR PATENT APPLICATION

This patent application claims benefit of pending prior U.S. Provisional Patent Application Ser. No. 60/901,218, filed Feb. 13, 2007 by Henry H. Fletcher for ACETABULAR DRILL GUIDE SYSTEM FOR CUP IMPLANTS (Attorney's Docket No. ORTHO-2 PROV), which patent application is hereby incorporated herein by reference.

### FIELD OF THE INVENTION

This invention relates to surgical procedures in general, and more particularly to total hip replacement procedures.

### BACKGROUND OF THE INVENTION

A total hip replacement is a reconstructive surgical procedure that is performed frequently by an orthopedic surgeon. A total hip replacement involves the placement of an acetabular cup in the acetabular socket of the patient, and the replacement of the femoral neck of the patient with a prosthesis winch terminates in a ball specifically designed to seat in the acetabular cup.

During the procedure, the acetabular socket is reamed out by the surgeon so as to create an enlarged recess to receive the acetabular cup. After the acetabular socket has been reamed out, the cup is inserted into the recess and adjusted as necessary to the proper angular orientation. Once deployed, the cup provides a new socket and lining for the acetabulum of the patient. See FIG. 1.

Insertion and placement of the cup by the surgeon is effected either by hand or by use of a hand tool that grips the cup. Once the cup is properly positioned in the acetabulum, the cup is fixed in the desired location by passing bone screws through the acetabular cup and into pre-drilled screw holes in the pelvic bone. The bone screws serve to hold the acetabular cup in the acetabulum until bone ingrowth provides permanent fixation.

When preparing to affix the acetabular cup, it is important that the screw holes be drilled in a precise alignment in order to ensure that the cup is secured in the proper position. Excessive angular misalignment, where the axis of the screw hole is not perpendicular to the wall of the hemispherical acetabular cup, can cause the head of the screw to protrude beyond the inner wall of the cup, thus interfering with full seating of the acetabular liner within the cup. Furthermore, off-axis placement of screws can also lead to shifting of the cup position, inasmuch as the eccentric contact between the screw head and the screw hole of the cup can exert a levering force on the cup when misaligned screws are tightened.

Due to the orientation of the hip joint, the reamed out acetabulum is relatively difficult tor the surgeon to access and visualize. Hence, after the acetabular cup is inserted into the acetabulum, it is difficult for the surgeon to maneuver and affix the cup at the desired location and with the proper orientation. This is due, in part, to the fact that most prior art placement tools are complicated, awkward and difficult to use, particularly when being used in a minimally invasive total hip arthoplasty procedure.

One of these prior art placement tools, a drill guide, is often used to assist in achieving accurate alignment when drilling the screw holes. The drill guide generally comprises a drill bushing on a handle, wherein the drill bushing is a sleeve that slideably accepts the drill bit used to prepare the bone for the screw. The exterior of the sleeve is sized to fit into the hole formed in the cup implant, where the head of the screw will later be seated. In some cases, a flexible drill shaft is often mated to the drill bit to enhance the surgeon's ability to drill at an angle into the side wall of the hemispherical acetabular cup.

After drilling the screw holes, the surgeon must select an appropriate screw length to affix the cup to the acetabulum. Ideally, this length is selected so as to maximize the depth that the screw will penetrate into the bone, without protruding past the distal surface of the bone. Selection of the screw length is often aided by use of a depth gauge. Prior art depth gauges generally comprise a probe, with depth markings and a flanged distal tip which a surgeon inserts into a previously-drilled hole. Upon pulling the depth gauge proximally, the flanged tip of the gauge hooks onto the distal rim of the bone, and the depth markings on the gauge can be read so as to determine the depth of the hole. Thus, the proper screw length can be selected.

The current state of instrumentation for drill guides, drills and depth gauges are difficult to use in a minimally invasive surgical procedure because of the reduced access and visibility attendant upon such procedures.

Thus, a significant need exists for an improved instrumentation system for securing an acetabular cap in an acetabulum during a minimally invasive total hip replacement procedure.

### SUMMARY OF THE INVENTION

It is, therefore, an object of the present invention to provide an improved system for securing an acetabular cup in the acetabulum.

In one form of the present invention, there is provided a drill guide system comprising:

a hemispherical cup having an opening therein;

a body having a distal end, a proximal end and a lumen extending between the distal end and the proximal end; and

a connector for connecting the body to the hemispherical cup so that the lumen at the distal end of the body is aligned with the opening in the hemispherical cup.

In another form of the present invention, there is provided a drill guide system comprising:

a surgical implant comprising a hole and a mount adjacent the hole;

a drill guide comprising an elbowed hollow tube having a distal end, a proximal end and a lumen extending between the distal end and the proximal end; and

a flexible drill comprising a distal end, a proximal end and a flexible shaft extending from the distal end to the proximal end;

wherein the distal end of the drill guide comprises a complementary mount for mating with the mount of the surgical implant so that the lumen at the distal end of the tube is aligned with the opening in the surgical implant.

In another form of the present invention, there is provided a depth gauge comprising:

a shaft having a distal end and a proximal end, the distal end of the shaft being split so as to form at least two dilatable arms, at least one of the arms having a laterally-extending flange having a surface lacing in the proximal direction, such that when the depth gauge is passed all the way through a hole in a bone, the flange can engage the far surface of the bone.

In another form of the present invention, there is provided a method for forming a hole in a bone adjacent to an acetabular cup of the sort having a hole therein, the method comprising:

providing a drill guide system comprising:
a hemispherical cup having, an opening therein;
a body having a distal end, a proximal end and a lumen extending between the distal end and the proximal end; and
a connector for connecting the body to the hemispherical cup so that the lumen at the distal end of the body is aligned with the opening in the hemispherical cup;

positioning the acetabular cup in the bone;

positioning the drill guide against the interior surface of the acetabular cup so that the opening in the hemispherical cup is aligned with the hole in the acetabular cup;

passing a drill through the lumen, through the opening in the hemispherical cup, through the hole in the acetabular cup, and into the bone.

### BRIEF DESCRIPTION OF THE DRAWINGS

These and other objects and features of the present invention will be more fully disclosed or rendered obvious by the following detailed description of the preferred embodiments of the invention, which are to be considered together with the accompanying drawings wherein like numbers refer to like parts and further wherein;

FIG. 1 is a schematic drawing showing a total hip prosthesis implanted in the hip joint;

FIGS. 2-5 are schematic drawings showing various drill guides formed in accordance with the present invention;

FIG. 6 is a schematic drawing showing a flexible drill and drill guide formed in accordance with the present invention;

FIGS. 7 and 8 are schematic drawings of drill tips for use with the flexible drill of the present invention;

FIG. 9 is a schematic drawing showing a flexible depth gauge and drill guide formed in accordance with the present invention;

FIG. 10 is schematic drawing showing another form of drill guide formed in accordance with the present invention;

FIG. 11 is a schematic drawing showing a drill guide and flexible drill of the present invention being used with a bone plate; and

FIG. 12 is a schematic drawing showing a drill guide and flexible drill of the present invention being used with an acetabular cup implant.

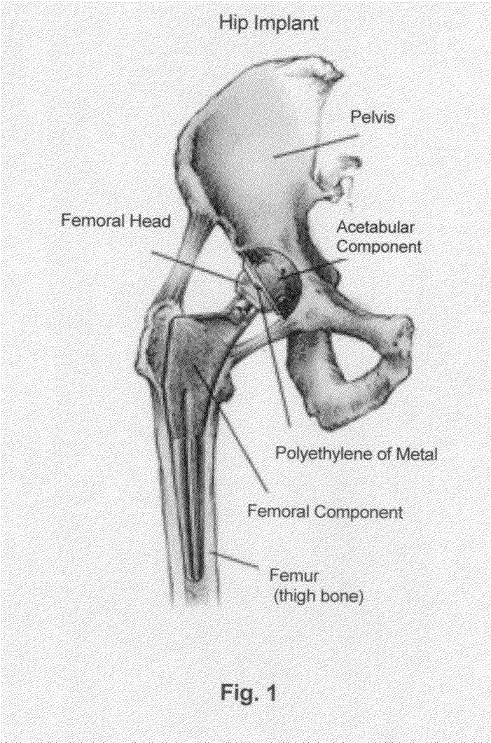

### DETAILED DESCRIPTION OF THE PRESENT INVENTION

The present invention provides an integrated instrumentation system which improves access and visualization during a total hip procedure, and particularly during a minimally invasive total hip procedure. More particularly, the present invention provides a drill guide, a flexible drill and a flexible depth gauge for use in securing an acetabular cup in the acetabulum.

### Drill Guide

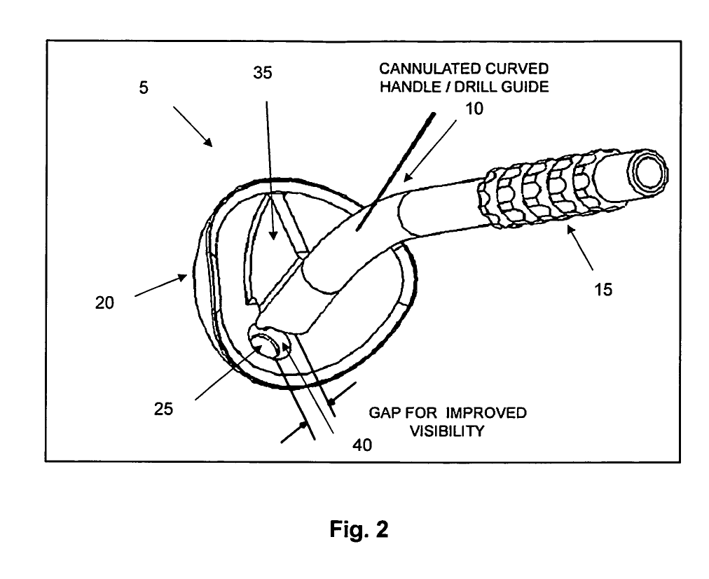

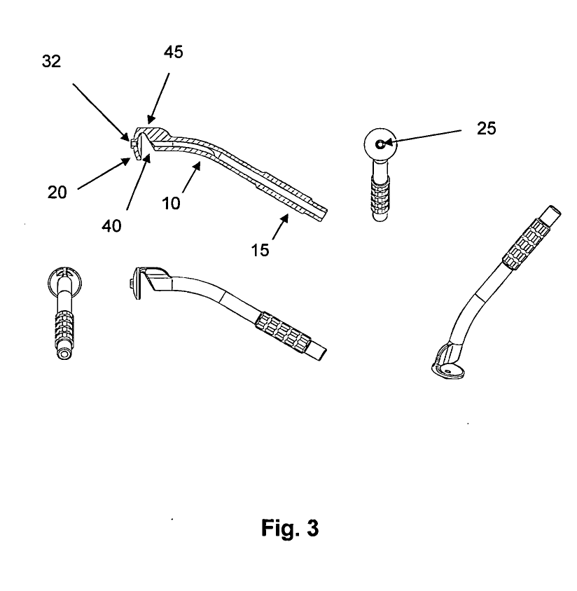

Looking now at FIGS. 2 and 3, there is provided a drill guide S comprising an elbowed, hollow tabs 10 and a handle 15 extending from the proximal end of hollow tube 10. Hollow tube 10 and handle 15 are preferably sized to accept a drill, as will be discussed in further detail below. Drill guide 5 also preferably comprises a hemispherical surface 20 disposed adjacent to the distal end of hollow tube 10.

As used herein with respect to surface 20, the terms “hemispherical”, “hemisphere” and the like are intended to denote a portion of the surface of a sphere or sphere-like surface. This portion might comprise one half of a sphere, but it is not intended to be necessarily limited to this meaning. Thus, when referring to surface 20, the terms “hemispherical”, “hemisphere” and the like may also denote a third of the surface area of a sphere, a quarter of the surface area of a sphere, or any other portion of the surface of a sphere. Accordingly, as used herein with respect to surface 20, the terms “hemispherical”, “hemisphere” and the like may be accurately replaced by the terms “semispherical”, “semisphere”, etc.

Hemispherical surface 20 is preferably sized and shaped so as to slidably fit into, and engage, the inner hemispherical wall of the acetabular cup implant. By sizing hemispherical surface 20 so that it engages the inner wall of the acetabular cup, the surgeon is assured that the axis of cannulation in hollow tube 10 and handle 15 is perpendicular to the wall of the acetabular cup at all times, thus ensuring that the drill will pass through the acetabular cup and into the pelvic bone on an axis which is precisely perpendicular to the hemispherically-shaped wall of the cup. Furthermore, providing cannulated tube 10 with an elbowed configuration can provide improved access where the angle of approach is dictated by anatomical structures which may not necessarily be linearly aligned. By way of example but not limitation, it may frequently be necessary to angulate an instrument by as much as 30 to 45 degrees as it moves from the angle of entering the body to the angle of addressing the acetabulum. The elbowed construction of tube 10 addresses this requirement.

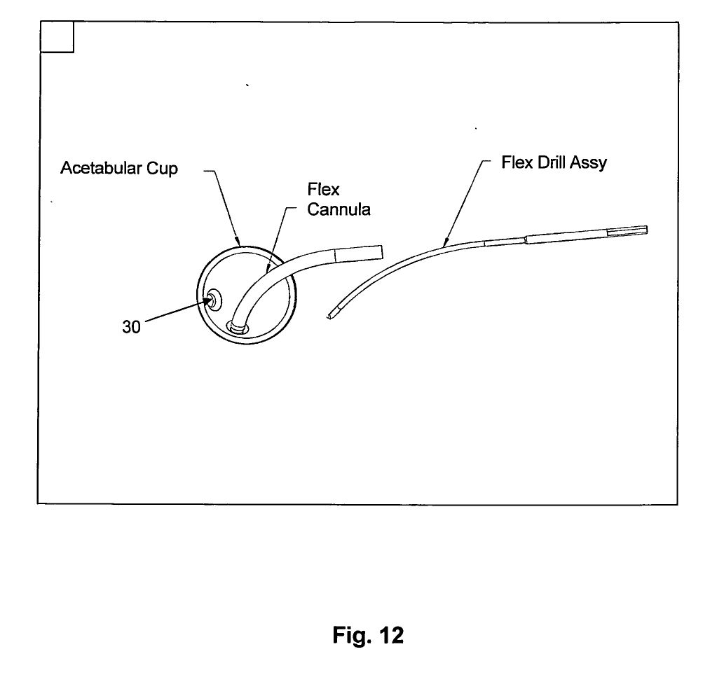

In one preferred embodiment, hemispherical surface 20 also comprises an opening 25 formed therein for alignment with a pre-drilled hole 30 of an acetabular cup implant (see FIG. 12). Hemispherical surface 20 further comprises a pilot guide 32 on the exterior of hemispherical surface 20. Pilot guide is aligned with opening 25, so that when pilot guide 32 is positioned in the hole of an acetabular cup implant, opening 25 will be aligned with the hole in the acetabular cup implant. Thus, a drill passing through opening 25 in hemispherical surface 20 is directed to pass through the hole in the acetabular cup and then into the acetabulum.

In one embodiment of the present invention, and as shown in FIG. 2, hollow tube 10 is connected to hemispherical surface 20 via a support bracket 35. Support bracket 35 extends from the interior of hemispherical surface 20 and engages the distal end of hollow tube 10. Support bracket 35 is preferably configured to engage the distal end of hollow tube 10 so that a gap 40 is created between the distalmost portion of hollow tube 10 and hemispherical surface 20. Gap 40 provides the surgeon with improved visibility during the procedure, by permitting the surgeon to watch as a drill, or screw passes out of hollow tube 10 and through opening 25 in hemispherical surface 20.

Hemispherical surface 20 may also be connected to hollow tube 10 via an integral bridge 45. As shown in FIG. 3, integral bridge 45 is preferably configured to transition into the distal end of hollow tube 10 so that a gap 40 is created between the distalmost portion of hollow tube 10 and hemispherical surface 20.

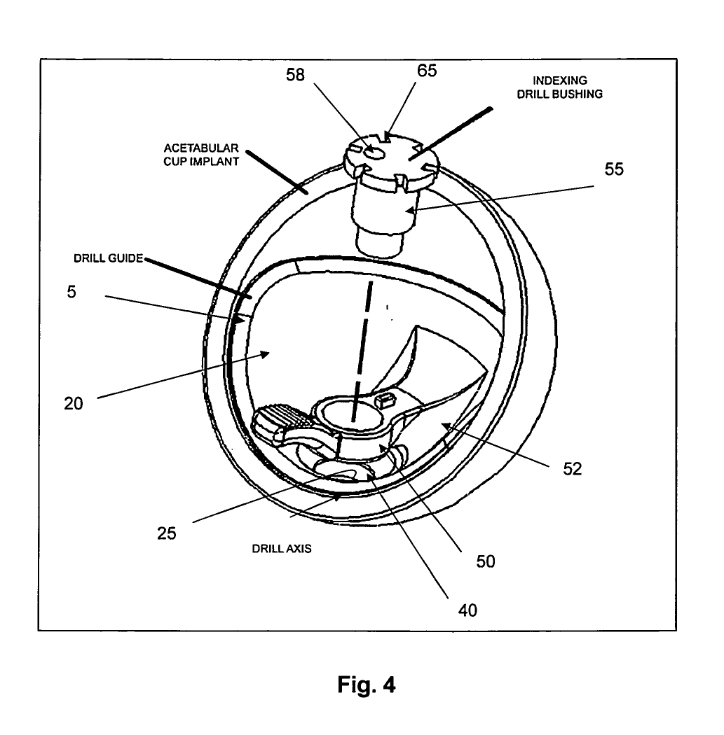

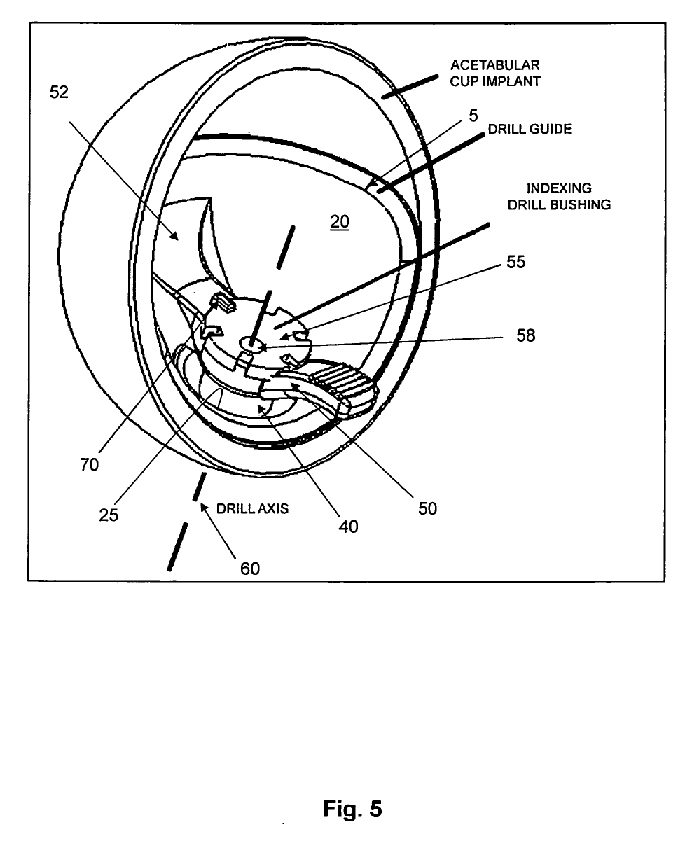

In another embodiment of the present invention, and as shown in FIGS. 4 and 5, drill guide 5 omits hollow tube 10 and provides, in its place, a housing 50 for receiving a drill bushing 55. Housing 50 is connected to hemispherical surface 20 by a bracket 52. Drill bushing 55 is mounted in housing 50 so that it sits adjacent to, but spaced slightly from, hemispherical surface 20. In other words, a gap 40 is provided between the distal end of drill bushing 55 and hemispherical surface 20 so as to permit the surgeon to visualize a drill passing from drill bushing 55 through opening 25 in hemispherical surface 20.

In addition, the drill hole 58 of drill bushing 55 may be parallel to, or even co-axially with, the center axis of drill bushing 55, or drill hole 58 may be set at an angle to the center axis of drill bushing 55, e.g., so as to increase the length of screw engagement.

In the case of an angled drill bushing, drill bushing 55 may be held in various rotational positions (e.g., 15 degrees off of the center axis) so as to adjust the relative angle of drill axis 60 (i.e., the center axis of drill hole 58) to drill guide 5. The various rotational positions may be pre-established by indexing the side of drill bushing 55 with notches 65 which are complementary to a mating feature 70 on housing 50.

In another embodiment of the present invention, at least one of hollow tube 10 and drill bushing 55 further comprise a window (not shown) so as to provide the surgeon with visual access between hollow tube 10 and hemispherical surface 20.

### Flexible Drill

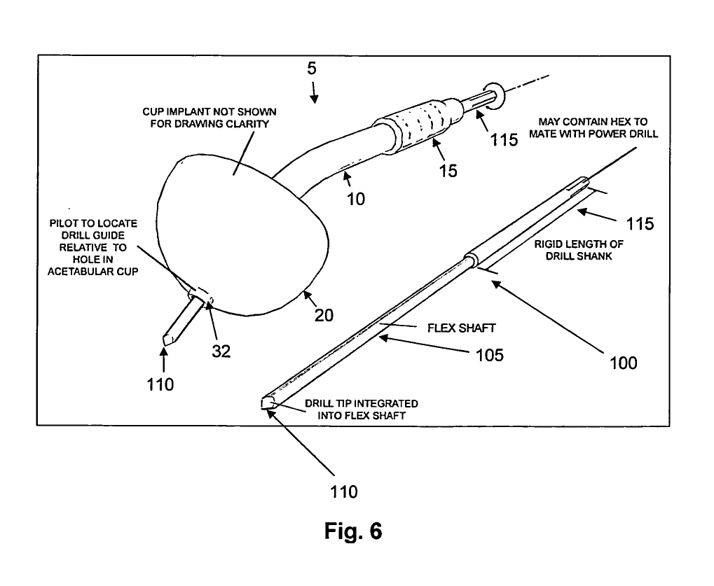

Looking now at FIG. 6, there is provided a flexible drill 100 for drilling a hole in the acetabulum. Drill 100 generally comprises a flexible shaft 105. Flexible shaft comprises a drill tip 110 integrated at its distal end and a rigid portion 115 integrated at its proximal end. Flexible drill 100 is able to bend along its length, so that it can drill a hole on an axis which is at an angle to the axis of a rotary drive tool. Preferably, flexible drill 100 is used with elbowed, cannulated hollow tube 10 and handle 15 of drill guide 5.

The flexible drill of the present invention is a significant improvement over existing prior art drills. More particularly, prior art drills comprise a rigid drill tip and a rigid drill shaft, coupled to a flexible shaft having a proximal end engaged in a driving instrument (e.g., a powered drill) which, is held by a surgeon. The distal rigid length of the drill tip and the rigid drill shank prevent passage of the drill through an elbowed cannulated handle of a drill guide, e.g., cannulated hollow tube 10.

In contrast, in the present invention, drill tip 110 is integrated directly into the distal end of flexible shaft 105 and rigid portion 115 of flexible drill 100 is greatly minimized, thus allowing passage through the elbowed cannulated handle of drill guide 5. Flexible drill 100 also comprises the minimal amount of rigidity at drill tip 110 (e.g., less than 0.50 inch, and preferably less than 0.20 inches). This allows drill tip 110 to pass through a curved cannula with a relatively small diameter.

It should be appreciated that although drill tip 110 is integrated with flexible shaft 105, drill tip 110 may still be removable from flexible shaft 105. For example, drill tip 110 may be secured to flexible shaft 105 with a secure, yet axially space-efficient, attachment mechanism (e.g., a latching bayonet).

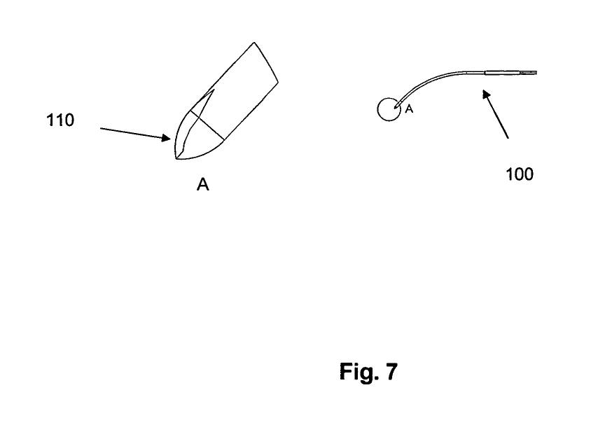

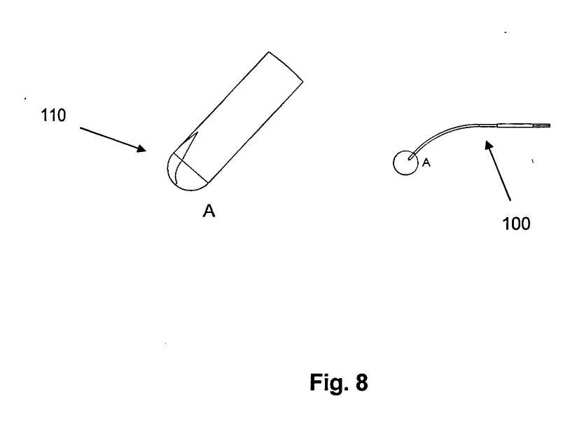

Looking next at FIGS. 7 and 8, drill tip 110 is configured so that it minimizes, or effectively eliminates, the “scuffing” created by a typical, conical drill tip as it passes through the drill guide. The novel drill, tip configurations of the present invention prevent debris from being “scuffed up” as the drill tip passes through the curved cannula of the drill guide.

Furthermore, in order to prevent “scuffing” during use, the drill is preferably inserted into the drill guide while not under power (i.e., not rotating).

In an alternative embodiment, flexible drill 100 may be inserted into drill guide 5 at the time of manufacture, with means incorporated into the drill guide/drill system for preventing the drill from being pulled proximally into the curved portion of the cannula, thus preventing drill tip 110 from “scuffing”.

In still another embodiment of the present invention, there is provided a flexible drill comprising a flexible shaft (or other driving means such as a series of links, rotationally keyed together yet pivoting at the joints of the links, e.g., in the manner of a ball-hex wrench) having a smaller diameter than the drill tip. By way of example but not limitation, the drill tip may be 0.125 inches and the flexible shaft may be 0.120 inches in diameter. By using a drill with a larger drill tip than the flexible shaft, the drill tip drills the intended diameter while debris formed from drilling can escape past the drill tip of the drill.

### Flexible Depth Gauge

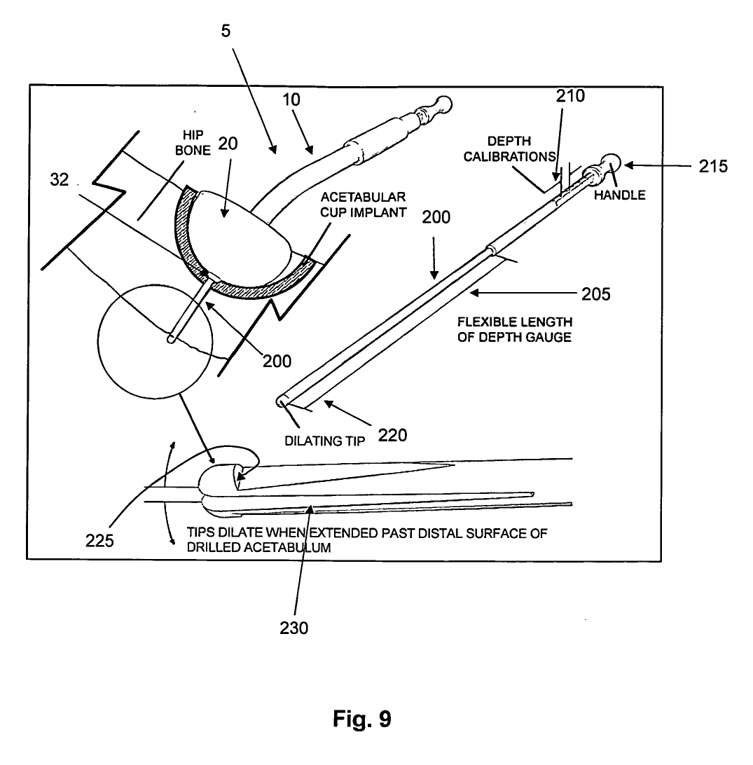

Looking next at FIG. 9, a calibrated flexible depth gauge 200 may be provided for measuring the depth of the screw hole. Depth gauge 200 composes a flexible shall 205 having a distal end and a proximal end. Depth gauge 200 is designed to be received in hollow tube 10 and handle 15 of drill guide 5, although it may also be used independently with this apparatus. The proximal end of depth gauge 200 comprises a plurality of depth markings 210 and a handle 215. In one embodiment, distal end of depth gauge 200 comprises a dilating tip 220. In one preferred construction, dilating tip 220 comprises two or more parallel elongated members which are capable of resiliently flexing their distal tips towards and away from one another. More particularly dilating tip 220 comprises a plurality of small fingers with at least one laterally-projecting flange 225 formed at the distal end of one or more of the fingers. When the flange extends beyond the distal opening of a drilled screw hole, the flange expands radially outward and engages the distal surface of the screw hole. The depth gauge may then be moved proximally so as to seat the depth gauge, and the surgeon can record the measurement and select the proper screw length.

In another embodiment of the present invention, the distal end of depth gauge 200 further comprises a ⅛ inch blunt-tip. In this embodiment, the depth gauge comprises graduated markings on the flexible shaft which may be used to gauge the depth of the drilled hole when a drill is prevented from completely breaking through the distal surface of the acetabulum, as will be discussed in further detail below.

In another embodiment, the flexible drill of the present invention may be used as the depth gauge. In this embodiment, the drill comprises depth markings on its shaft to enable a surgeon to gauge the depth of the drilled hole while drilling occurs, or to gauge the depth of the drilled hole after the screw hole has been drilled.

Once the depth of the screw hole has been determined, the appropriate length screw is placed into each of the screw holes in the acetabular cup, engaging to a depth nearly adjacent to the distal end of the drilled holes, with the head of the screws anchoring the cup firmly to the acetabulum.

### Additional Embodiments Of The Drill Guide

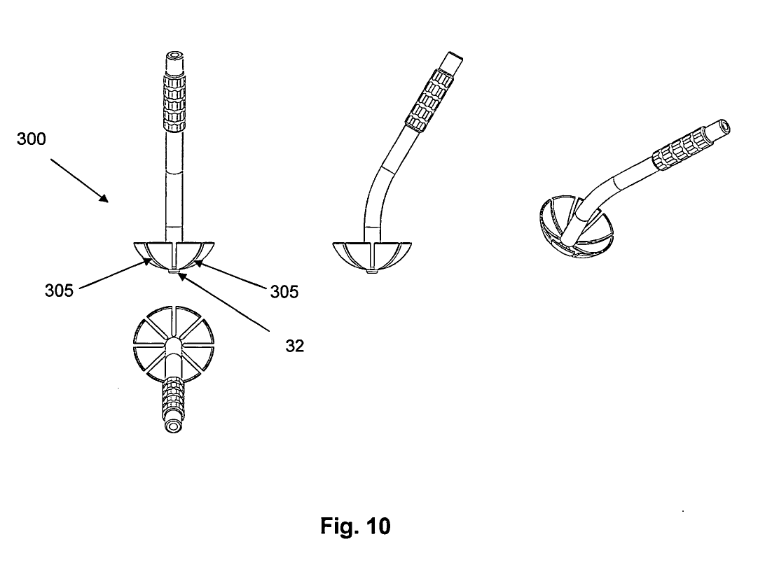

Looking now at FIG. 10, there is provided a drill guide 300. Drill guide 300 is similar to drill guide 5 discussed above, however, hemispherical surface 20 comprises a series of radial cuts 305 which provide the hemispherical surface with increased flexibility. The flexible nature of the hemispherical surface enables the drill guide of the present embodiment to be used with a variety of acetabular cap implant sizes. Additionally, the flexible nature of the hemispherical cup allows the surgeon to angulate the drill off of the center axis in order to aim the drill toward the best available bone to seat a screw.

This construction may be utilized with rigid hollow tube 10, flexible hollow tube 10 or housing 50, and/or with a rigid drill or a flexible drill, depending on the access and visibility needed to secure the acetabular cup implant in the acetabulum.

In another embodiment of the present invention, there is provided a drill guide with a small hemispherical dome 20 (FIG. 3) or, alternatively, hemispherical dome 20 may be cut back to the point where it effectively provides a thin radial arm supporting a pilot guide. The small hemispherical dome (or radial arm) eliminates, or at least minimizes, the need for multiple sizes of hemispherical surfaces for mating with each acetabular cup implant size. The small hemispherical dome (or radial flange) also allows limited angulation to the drill guide. More particularly, many surgeons desire to “aim” the drill at a more solid bony area, which may or may not be aligned with the perpendicular axis of the acetabular cup implant, but instead may be 15 degrees or more off-axis. With the smaller hemispherical dome, or the radial flange, the surgeon is able to aim the drill oil of the perpendicular axis of the acetabular cup implant.

In another embodiment, the hemispherical surface of the drill guide may be asymmetrical, or truncated on one or more edges, to allow the guide to seat over a screw hole in a cup without significantly overlapping the cup, and to facilitate insertion into the small incision in the patient.

### Use of Drill Guide and Flexible Drill for Trauma Implants

In another embodiment, the drill guide and flexible drill of the present invention may be utilized with a trauma implant.

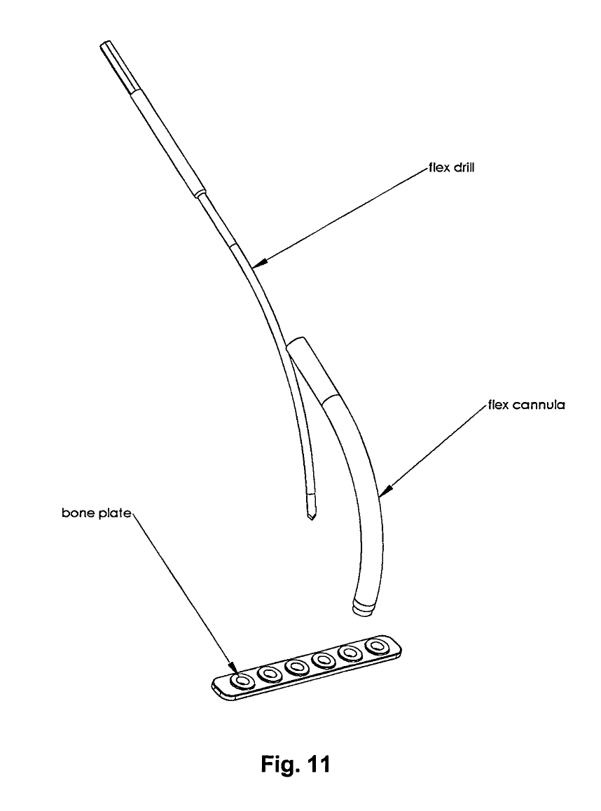

In this embodiment, the distal end of drill guide 5 features threads, or other stabilization or attachment means, which mate with the threads (or counterpart stabilization or attachment means) of holes in a locking screw plate, or a bone plate. The flexible drill permits drilling of bone perpendicular to the plate, permitting a locking screw to be placed with a universal tip screw driver. Conventionally, with prior art drill guides and locking plates, a surgeon may be forced, to choose a sub-optimal position for a plate because drilling a hole perpendicular to the plate would require excessive dissection of soft tissues in order to permit a conventional drill and a conventional drill guide to be used. See FIG. 11.

Looking now at FIG. 12, the drill guide and flexible drill of the present invention may be used for placement of adjuvant screw fixation of acetabular shells. More particularly, acetabular cup implants could be manufactured with smooth, beveled screw holes. The flexible drill and drill guide would allow for use of locking screw technology in acetabular implants similar to the threaded locking screws for bone plates. In this embodiment, the distal end of drill guide comprises male threads which mate with the female threads of screw holes manufactured with locking screw threads in the acetabular cup implants. A surgeon can choose to drill through the acetabular implant with a “locked” position by screwing the distal tip of the drill guide onto the acetabular screw hole threads. Alternatively, the surgeon may choose to use the drill guide to drill a screw hole, as discussed above.

The surgeon can also choose to use a locking screw which engages is the acetabular screw hole as a rigid construct. This is useful in osteoporotic bone where a standard non-locking screw may not gain adequate purchase to pull the acetabular shell tight to the bone. A locking screw acts much as fixed pegs, which is another form of adjuvant fixation manufactured in acetabular cups to prevent motion for bone ingrowth to occur.

### Flexible Drill with Torque Sensor

In one embodiment of the present invention, the flexible drill is provided with a drill torque sensor which stops the drill from spinning as the drill nears penetration. The drill torque sensor is a safety feature which helps avoid injury to neurovascular structures.

Additionally, having to retract the drill and insert a depth gauge is a frequent source of frustration in the operating arena, especially when the surgeon has a difficult time finding the distal surface of the drilled hole with the depth gauge. The drill torque sensor saves time by permitting a surgeon to measure the depth of a drill hole for subsequent screw placement, without retracting the drill, and inserting a drill guide with a depth gauge.

The drill torque sensor also may be used to drill and place the proximal interlocking screws of an intramedullary rod. More particularly, many of the manufacturers of intramedullary hip screws and intramedullary rods for the femur, tibia, and humerus have outrigger attachments for the implant insertion tools. The surgeon utilizes the outriggers as a drill guide to ensure a drill can be passed through the bone, through a hole in the implant, and through the bone on the far side of the implant hole, thereby “interlocking” the implant to the bone. A torque sensor feature facilitates this step by making drill removal and depth gauge insertion unnecessary to choose the proper length screw to use.

### Flexible Drill with a Torque Etching Device

In another embodiment of the present invention, there is provided a torque latching device, e.g., a cranial perforator, with a driving drill device which does not completely perforate the distal bone surface of the acetabulum. The torque latching device provides an easy and reliable means for gauging the depth of the drilled hole with a probe or with the drill itself (e.g., with depth markings on the flexible shaft, as discussed above).

In one embodiment of the present invention, the torque latching device is used as follows: as the drill tip begins to penetrate the distal surface of the bone, the change in torque triggers the drill to disengage the drill bit.

Alternatively, the change in torque may be accomplished electronically in ways well known in the art. More particularly, the drill, or drill controller may comprise current or load-monitoring circuitry, such as the circuitry typically used with brushless DC motors.

In another embodiment, a cranial perforator may be used solely with the drill, rather than a separate mechanical device, to stop drilling.

### Manufacturing

The drill guide of the present invention preferably comprises plastic. More particularly, hemispherical surface 20 preferably comprises a disposable plastic component. The plastic composition prevents the drill guide from scratching the acetabular cup implant.

Depth gauge 200 also preferably comprises a plastic component.

The instrumentation system (i.e., drill guide 5, drill 100 and depth gauge 200) may be supplied to a surgeon in a sterile-disposable unit.

### Modifications

While the present invention has been described in terms of certain exemplary preferred embodiments, it will be readily understood and appreciated by those skilled in the art that it is not so limited, and that many additions, deletions and modifications may be made to the preferred embodiments discussed herein without departing from the scope of the invention.

What is claimed is:

1. A drill guide system comprising;
a hemispherical cup having an opening therein;
a body having a distal end, a proximal end and a lumen extending between the distal end and the proximal end; and
a connector for connecting the body to the hemispherical cup so that the lumen at the distal end of the body is aligned with the opening in the hemispherical cup.

2. A drill guide system according to claim 1 wherein a gap is provided between the distal end of the body and the opening in the hemispherical cup.

3. A drill guide system according to claim 1 wherein the body comprises a hollow tube.

4. A drill guide system according to claim 1 wherein the hollow tube is substantially rigid.

5. A drill guide system according to claim 4 wherein the hollow tube is elbowed along its length.

6. A drill guide system according to claim 3 wherein the hollow tube is flexible along at least a portion of its length.

7. A drill guide system according to claim 1 wherein the hemispherical cup, body and connector are formed integral with one another.

8. A drill guide system according to claim 1 wherein the body comprises a housing having a relatively short lumen.

9. A drill guide system according to claim 8 wherein the lumen of the housing receives a drill bushing therein.

10. A drill guide system according to claim 9 wherein the drill bushing comprises a drill hole which is set at an angle to the longitudinal axis of the drill bushing.

11. A drill guide system according to claim 9 wherein the drill bushing is indexed relative to the housing.

12. A drill guide system according to claim 1 wherein the hemispherical cup further comprises a pilot guide on its exterior surface for seating in a hole of an acetabular cup implant.

13. A drill guide system according to claim 1 wherein the system further comprises a flexible drill comprising a distal end, a proximal end and a shaft extending from the distal end to the proximal end, wherein at least a portion of the shaft is flexible.

14. A drill guide system according to claim 13 wherein the distal end of the flexible drill comprises a frustoconical tip with a cutting flute.

15. A drill guide system according to claim 13 wherein the distal end of the flexible drill comprises a hemispherical front tip with a cutting flute.

16. A drill guide system according to claim 13 wherein the flexible drill further comprises depth markings on the shaft.

17. A drill guide system according to claim 1 wherein the hemispherical cup is slotted so as to create a plurality of petals.

18. A drill guide system according to claim 1 wherein the hemispherical cup is flexible.

19. A drill guide system comprising:
a surgical implant comprising a hole and a mount adjacent the hole;
a drill guide comprising an elbowed hollow tube having a distal end, a proximal end and a lumen extending between the distal end and the proximal end; and
a flexible drill comprising a distal end, a proximal end and a flexible shaft extending from the distal end to the proximal end;
wherein the distal end of the drill guide comprises a complementary mount for mating with the mount of the surgical implant so that the lumen at the distal end of the tube is aligned with the opening in the surgical implant.

20. A drill guide system according to claim 19 wherein the surgical implant is an acetabular cup.

21. A drill guide system according to claim 19 wherein the surgical implant is a bone plate.

22. A drill guide system according to claim 19 wherein the first mount and the complementary mount both comprise screw threads.

23. A depth gauge comprising:
a shaft having a distal, end and a proximal end, the distal end of the shaft being split so as to form at least two dilatable arms, at least one of the arms having a laterally-extending flange having a surface facing in the proximal direction, such that when the depth gauge is passed all the way through a hole in a bone, the flange can engage the far surface of the bone.

24. A method for forming a hole in a bone adjacent to an acetabular cup of the sort having a hole therein, the method comprising:
providing a drill guide system comprising:
a hemispherical cup having an opening therein;
a body having a distal end, a proximal, end and a lumen extending between, the distal end and the proximal end; and
a connector for connecting the body to the hemispherical cup so that the lumen at the distal end of the body is aligned with the opening in the hemispherical cup;
positioning the acetabular cup in the bone;
positioning the drill guide against the interior surface of the acetabular cup so that the opening in the hemispherical cap is aligned with the hole in the acetabular cup;
passing a drill through the lumen, through the opening in the hemispherical cup, through the hole in the acetabular cup, and into the bone.

25. A method according to claim 24 wherein passing a drill through the lumen, through the opening in the hemispherical cup, through the hole in the acetabular cup, and into the bone includes using a torque sensor while drilling so as to avoid drilling completely through the bone.
